(19) United States
(12) Patent Application Publication
(10) Pub. No.: US 2022/0214284 A1
(43) Pub. Date: Jul. 7, 2022

(54) APPARATUS AND METHODS FOR ASSAYING A LIQUID SAMPLE

(71) Applicant: Redcoat Solutions, Inc., Rockingham, VA (US)

(72) Inventors: William John HALL, Harrisonburg, VA (US); Andy STURMAN, San Diego, CA (US)

(21) Appl. No.: 17/525,447

(22) Filed: Nov. 12, 2021

Related U.S. Application Data
(60) Provisional application No. 63/112,703, filed on Nov. 12, 2020.

(51) Int. Cl. G01N 21/84; G01N 33/543

(52) U.S. Cl. CPC G01N 21/8483; G01N 33/558; G01N 33/54386

(57) ABSTRACT

Devices, systems, and kits for assaying a liquid sample include a reader and a test strip. The test strip may comprise a disposable cassette. The system may include a test strip mounting structure configured to receive the cassette in a predetermined orientation. The apparatus includes a processor and memory, together configured to conduct an assay of the liquid sample. The apparatus may include a display configured to generate a pattern encoding data related to a result of the assay.

### RELATED APPLICATIONS

This application claims priority to U.S. Provisional Application 62/112,703 filed on Nov. 12, 2020, the disclosure of which is incorporated by reference herein in its entirety.

### BACKGROUND

It is often desirable in medicine or the biological sciences to be able to determine the presence or concentration of a particular target substance in a biological and/or liquid sample. While many methods of performing such assays are known, conventional methods often require the use of expensive equipment. This can meaningfully limit access to and cost effectiveness of certain scientific and/or professional practices that require, or are rendered more effective through, the use of such assays.

Devices and systems that can be used in the home by untrained consumers have been developed. These include, for example, commercially available pregnancy and ovulation test devices such as the Clearblue® Fertility Monitor. Such test devices generally require complex use instructions and are subject to inadvertent user error that can interfere with obtaining accurate test results. Accordingly, systems and methods for performing assays at a reduced cost and/or with increased convenience are desirable.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

In some embodiments, a device for assaying a liquid sample is provided. A liquid sample assay device comprising a first housing portion, a second housing portion, a test strip mounting location associated with at least one of the first housing portion and the second housing portion, a user operable coupling configured to selectively allow open and closed configurations of the first housing portion with respect to the second housing portion, wherein the coupling is configured to allow a user to place the first housing portion and the second housing portion in the open configuration to the expose the test strip mounting location, place a test strip at the test strip mounting location, and place the first housing portion and the second housing portion in the closed configuration for performing an assay of the test strip.

A device for assaying a liquid sample, the device comprising a housing configured to receive a test strip in a predetermined orientation such that a portion of the test strip is disposed within the apparatus and a portion of the test strip extends outside of the apparatus at least one sensor in the housing configured to generate a signal indicative of a change in a characteristic of the sample strip, and a display configured to generate a machine-readable pattern encoding data related to the signal.

A test strip cassette for performing a liquid assay, the test strip cassette comprising a housing; a lateral flow test strip inside the housing; an opening in the housing exposing a portion of the lateral flow test strip; and a first registration feature associated with the opening configured to align a sensor in a test strip reader with the exposed portion of the lateral flow test strip.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIG. 1 is a block diagram of a system for assaying a liquid sample according to some example embodiments;

FIG. 2A shows a reader for testing an assay, according to some example embodiments;

FIG. 2B illustrates several components of the reader of FIG. 2A, according to some example embodiments;

FIG. 2C is a block diagram of a test strip for assaying, according to some example embodiments;

FIG. 2D is an exemplary graph of signals provided by sensors of the reader of FIGS. 2A through 2C, according to some example embodiments;

FIG. 3A shows a hand-held probe for receiving data related to an assay from a reader, according to some example embodiments;

FIG. 3B illustrates several components of the probe of FIG. 3A, according to some example embodiments;

FIG. 4 is a block diagram of example components of the reader of FIG. 1, according to some example embodiments;

FIG. 5 is a block diagram of example components of the probe of FIG. 1, according to some example embodiments;

FIG. 6 is a block diagram of an example embodiment wherein the probe of FIG. 4 is configured to receive the reader of FIG. 5, according to some example embodiments;

FIG. 7 is a block diagram of a single housing integrating some components of the reader of FIG. 4 and some components the probe of FIG. 5, according to some example embodiments;

FIG. 8 is a block diagram for an example embodiment of the single apparatus and/or housing of FIG. 7, configured to receive a cassette comprising a test strip to be assayed, according to some example embodiments;

FIG. 9 illustrates a perspective view of an example embodiment of an apparatus configured to receive a cassette comprising a sample to be assayed, according to some example embodiments;

FIG. 10 illustrates a perspective view of the apparatus of FIG. 12 having the cassette disposed therein, according to some example embodiments;

FIG. 11 illustrates a system comprising an apparatus configured to receive a cassette comprising a sample to be assayed, according to some example embodiments;

FIG. 12 illustrates another system comprising another apparatus configured to receive a cassette comprising a sample to be assayed, according to some example embodiments;

FIGS. 13A and 13B illustrate various example screen shots of a display of a device of a system for assaying a sample in a cassette, according to some example embodiments;

FIG. 14A illustrates a perspective top view of a device configured to receive a cassette comprising a sample to be assayed in a closed configuration, according to some example embodiments;

FIG. 14B illustrates a perspective bottom view of the device of FIG. 14A;

FIG. 15A is a perspective top view of a test strip cassette, according to some example embodiments.

FIG. 15B is a side cross section of a test strip cassette, according to some example embodiments.

FIG. 16 illustrates a perspective view of the device of FIG. 14 in an open configuration, according to some example embodiments;

FIG. 17 illustrates another perspective view of the device of FIG. 14 in an open configuration with a battery cover installed, according to some example embodiments;

FIG. 18 illustrates another perspective view of the device of FIG. 14 in an open configuration with a test strip placed on the test strip mounting location, according to some example embodiments;

FIG. 19 is an exploded view showing components of the device of FIG. 14, according to some embodiments;

FIG. 20 is a cutaway top perspective view of the device of FIG. 14 in an open configuration, according to some example embodiments;

FIG. 21 is a cutaway bottom view of the device of FIG. 14 in an open configuration, according to some example embodiments;

FIG. 22 is a cutaway side view of the device of FIG. 14 in an open configuration, according to some example embodiments;

FIG. 23 is a cutaway side view of the device of FIG. 14 in a closed configuration, according to some example embodiments;

FIG. 24 is a close-up view of the cassette and mounting structure of FIG. 21;

FIG. 25 is a close-up of the cassette and mounting structure of FIG. 23.

FIG. 26 illustrates a flowchart of another method for testing an assay and reading out a result of such testing, according to some example embodiments.

FIG. 27 illustrates a flowchart of a method for testing an assay and reading out a result of such testing, according to some example embodiments;

FIG. 28 illustrates a flowchart of another portion of a method for testing an assay and reading out a result of such testing, according to some example embodiments.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

Definitions

Analyte: The term analyte refers to any substance or chemical constituent of a fluid such as but not limited by water, alcohol, or any other diluent. Analytes include without limitation naturally occurring substances, artificial substances, metabolites, and reaction products, such as proteins, carbohydrates, nucleic acids, fats, hormones, antigens, antibodies, amino acids, vitamins, components of organisms, components of cells of organisms, including any molecular components of animals, plants, viruses, parasites, bacteria, fungi, and/or chemical compounds or products produced or consumed by such organisms or the cells thereof. The term analyte also includes any drug or pharmaceutical composition that has been or may be introduced into an organism.

App: An app is a software application program. The term app is usually applied to a software program that is executable on smartphone hardware running smartphone operating systems such as iOS and Android. These are often referred to as mobile apps. Although a mobile app is generally designed for operation on mobile devices, a mobile app can be executed on non-mobile devices such as desktop or laptop computers that are running an operating system compatible with the mobile app.

Final Assay Result Output: A final assay result output is a presentation of an assay result that constitutes actionable or otherwise directly useful information for a user of the assay. A final assay result output includes without limitation a binary output indicating the presence or absence of an analyte or a numerical estimation of an amount of analyte present in a sample. A test error indication may also be a final assay result output.

Processor: A processor is an electronic circuit configured to retrieve instructions from a memory and execute one or more arithmetic, logic, data storage, and/or data output operations defined by the retrieved instructions. A processor may execute these operations sequentially or concurrently. A processor may be any conventional general-purpose single- or multi-chip processor found in consumer devices such as personal computers, laptop computers, smartphones, and the like. In addition, a processor may be any conventional special purpose processor such as a digital signal processor, a graphics processor, or a microcontroller.

Processing Circuitry: Processing circuitry is any arrangement of electrical or electronic components that is configured to receive as input one or more analog or digital signals or data and generate as output one or more analog or digital signals or data in response. Processing circuitry may comprise one or more buffers, voltage dividers, filters, logic gates, adders, and the like. A processor comprises processing circuitry and processing circuitry may include one or more processors and memory. However, processing circuitry does not necessarily implement all or any of the functions associated with a processor as set forth above.

E-Paper: A commercially available low power display technology with bi-stable pixels that can maintain an image written to the display essentially indefinitely without consuming any power.

QR code: A two-dimensional array of dark squares on a light background that encodes information having a configuration standardized in ISO/IEC 18004.

Software and Program: The term software or program refers to instructions stored in a memory in machine-readable form, human-readable form, or both that are executable by a processor when compiled into a machine-readable form. Software may be written in a variety of programming languages such as but not limited to the various versions of C and JavaScript. Depending on the environment of use, software may be also called firmware.

Browser and Web Page: A browser is a computer program that provides functionality to a computer for executing syntax that may be contained in web pages. The computer may be connected to a computer network, and the network may be, and usually will be, the Internet. As used herein, browsers and web pages together provide functionality to a computer connected to a network (e.g. the Internet) at least sufficient to request, retrieve, and display at least some network resources including web pages themselves, and to execute at least some links contained within or referred to in retrieved web pages to retrieve other web pages specified with the links. Web pages may include references such as uniform resource locators (URLs) and/or universal resource identifiers (URIs) to other network resources that contain images or other data that is retrieved by the browser from the network or from a cache memory when executing the web page, and may also include and/or reference programs, libraries, style sheets, scripts, and the like which are run by the browser when executing a web page. Any of these items that are accessed, used, and/or retrieved during browser engine execution of web page syntax are considered to be included as a component of the “web page” as that term is used herein. Examples of browsers include, but are not limited to, Internet Explorer and Edge distributed by Microsoft, and Chrome distributed by Google. Example web page syntax that can be executed by browser engines is the various versions of HyperText Markup Language (HTML) promulgated by the World Wide Web Consortium (W3C) and client-side scripting languages such as JavaScript.

Server: The term server refers to a software program that is configured to cause computer hardware to respond to client access requests to use or retrieve network resources. The term server also refers to computer hardware that is executing server software. A “server” will often have a functional term applied to it indicating a functionality of the server software and/or hardware such as print server, file server, or web server.

Internet: The globally interconnected system of computers and computer networks that evolved from ARPANET and NSFNET over the late 1980s and early 1990s that may utilize TCP/IP network communication protocols.

Cloud Services: A collection of network resources configured to support the operation of one or more software programs executing on one or more user devices such as desktop or laptop computers or smartphones.

Network Resource Identifier: A definition of a network resource (e.g., by storage location and filename) that is used by client computers to specify a network resource in access requests issued to the network by the client computers. A network resource identifier may be analogized to a “location” of a network resource such as an image or a web page. Currently, when the network is the Internet, network resource identifiers are known as URLs that are formatted in accordance with RFC 3986 of the Internet Engineering Task Force (IETF). For the purposes of this disclosure, any format for specifying a network resource in client access requests issued to a network is within the definition of the term network resource identifier. A network resource identifier, including URLs as currently defined on the Internet, may further include data in addition to data identifying the network resource that a server hosting the network resource associated with the network resource identifier may use for other purposes beyond identifying the requested network resource.

Web Site: A collection of network resources including at least some web pages that share a common network resource identifier portion, such as a set of web pages with URLs sharing a common domain name but different pathnames.

Web Server: A server that includes functionality for responding to requests issued by browsers to a network, including, for example, requests to receive network resources such as web pages. Currently, browsers and web servers format their requests and responses thereto in accordance with the HyperText Transfer Protocol (HTTP) promulgated by the IETF and W3C. In some embodiments, a web server may also be a content server.

Network Resource: A web page, file, document, program, service, or other form of data or instructions which is stored on a network node and which is accessible for retrieval and/or other use by other network nodes.

Algorithm: A connected sequence of two or more data processing acts. Software programs are implementations of algorithms.

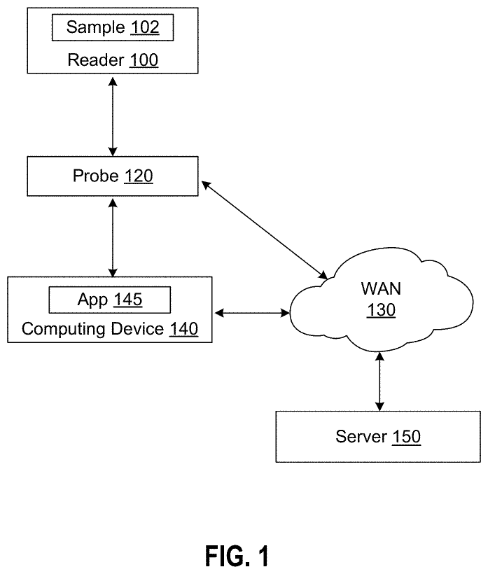

FIG. 1 shows a block diagram of a system for performing an assay and outputting a result of the assay procedure, according to some example embodiments. The system can include any one or more of a reader 100, a probe 120, a computing device configured to run an app 145 and a server 150, which may be one or more of a node of a local area network, a web server, and/or a component or collection of components hosting Internet connected cloud services.

As will be described in more detail below in connection with one or more other figures, reader 100 may be configured to receive a test strip 102 containing a sample thereon and perform an assay of the sample by generating a signal or other form of test data indicative of the presence and/or estimated amount or concentration of an analyte in the sample at one or more locations on strip 102 at one or more instants in time. The test strip 102 may comprise a porous carrier and one or more reagents forming a lateral flow immunoassay test strip. The construction and functionality of such strips are well known, with one example being the test strips described in U.S. Pat. No. 10,823,726 assigned to the applicant, which is incorporated by reference herein in its entirety. A wide variety of technologies for reading lateral flow assay test strips are available which may be optical, resistive, or magnetic in nature.

The reader 100 may also output an indication of the presence and/or estimated amount or concentration of the analyte to one or more other devices, for example, probe 120, computing device 140 and/or server 150. Typically, the front-end sensing technology of the reader 100 generates a series of raw data points indicative of a property of the test strip 102 at one or more times during the assay process. This data may be pre-processed with filters and the like if desired and is then further processed to generate a final assay output. This further processing to generate the final assay output may involve baseline corrections, comparisons to thresholds, linear or non-linear fitting, calibration, and the like to generate a final assay output. The final assay output may comprise text or other binary indication of the presence or absence of the analyte, may be a numerical estimate of the concentration of the analyte in the sample, or may be another mathematical manipulation of the raw and/or pre-processed data to produce an output that conveys the assay result information in a manner actionable or otherwise useful to a user of the assay.

In some embodiments, the reader 100 and test strip 102 are integrally constructed as a single use disposable device. To reduce production costs for a disposable product, in some embodiments, reader 100 may be configured to sense a level of a target substance at one or more locations on sample strip 102 and at one or more instants in time but not configured to directly communicate those sensed levels to computing device 140 and/or computing device 150 via, for example, a Bluetooth or WiFi connection and may also not be configured to determine whether one or more conditions of the testing are satisfied to generate and/or output a final assay result. Producing a reader 100 in this manner allows reader 100 to be manufactured with reduced functionality and requiring fewer components components and is, therefore, better suited for disposable, single-use applications. However, the present disclosure is not so limited, and reader 100 can in some embodiments be configured to directly communicate those sensed levels to computing device 140 in a similar manner as probe 120 is configured, as described anywhere in this disclosure.

The probe 120 may be configured to receive an output from the reader 100 indicative of the presence and/or sensed level(s) of the analyte on strip 102. In some embodiments, the received output may be one or more raw or minimally processed data values generated by the sensor technology used by the reader such as strip reflectivity or conductivity values measured by the reader at different time points and/or different locations on the test strip 102. In some embodiments, the reader 100 output received by the probe 120 may be a single processed binary indication such as “present” or “not present” or a single analyte concentration estimate generated by processing circuitry in the reader 100. The probe 120 may further communicate one or more indications of the sensed presence or level(s) of the analyte on test strip 102 to one or more other devices, for example, computing device 140 and/or server 150. In some embodiments, probe 120 is configured to directly communicate the reader 100 output or a processed reader output to a separate computing device 140. This communication may, for example, be over a wireless connection implementing a standard protocol such as Bluetooth. In some embodiments, probe 120 is configured to indirectly communicate the reader 100 output to computing device 140 via, for example, wide area network 130, which may comprise a portion of the Internet and/or any other suitable wired or wireless network or portions thereof. This may be performed via a WiFi connection to a router for communication over the Internet. In some embodiments, the probe 120 may function essentially as a relay device that does not perform significant data processing on the output that the probe 120 receives from the reader 100. In some embodiments, probe 120 is configured to process raw or pre-processed data received by the reader to generate a final assay output using processing circuitry located in the probe 120 and transmit this processed output to computing device 140 and/or server 150. The probe 120 may additionally or alternatively be configured to display one or more indications of the sensed level(s) of the target substance on sample strip 102 from the reader 100 and/or processed information derived at least partly therefrom and/or a final assay output. Accordingly, in such embodiments, while not shown in FIG. 1, the probe 120 can further include a display configured to present information externally that may be human readable and/or machine readable.

Computing device 140 may be configured to execute an app 145 which may be configured to receive, process, and/or output raw or processed data of such an assay performed by reader 100. In some embodiments, computing device 140 can be a smartphone, tablet, personal computer, server, or any other suitable computerized user terminal. Computing device 140 may be configured to receive, directly or indirectly, one or more indications of the received sensed level(s) of the analyte on test strip 102 from the probe 120. Based at least in part on those indications, app 145 may be configured to determine whether one or more conditions of the testing are satisfied and/or to display a result of such a determination to a user. In some embodiments, app 145 is additionally or alternatively configured to display one or more indications of the sensed level(s) of the target substance on test strip 102 and/or processed information derived at least partly therefrom. Accordingly, in such embodiments, while not shown in FIG. 1, computing device 140 can further include a display configured to present information externally that may be human readable and/or machine readable.

In some embodiments, at least part of the processing of the indications of the received sensed level(s) of the target substance on sample strip 102 and/or the determination of whether one or more conditions of the testing are satisfied can be performed remote from app 145 and/or of computing device 140, for example, by a remote computing device 150, which may be a server, a cloud computing system, or any other suitable computing device.

In some embodiments, computing device 140 and/or server 150 is/are configured to receive the one or more indications of the received sensed level(s) of the target substance on sample strip 102 directly or indirectly from probe 120 and determine whether one or more conditions of the testing are satisfied and/or display one or more indications of the sensed level(s) of the target substance on sample strip 102, and/or processed information derived at least partly therefrom, to a user.

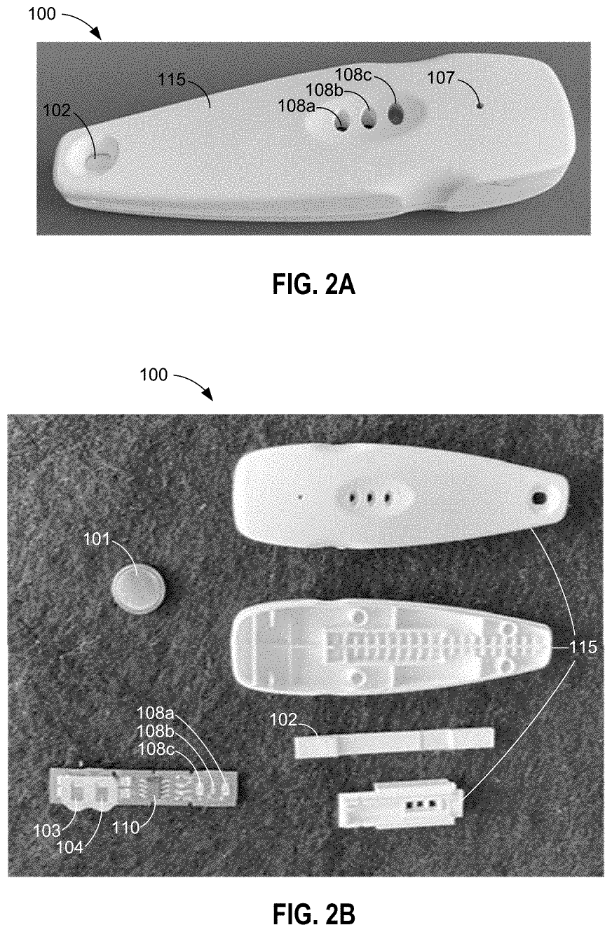

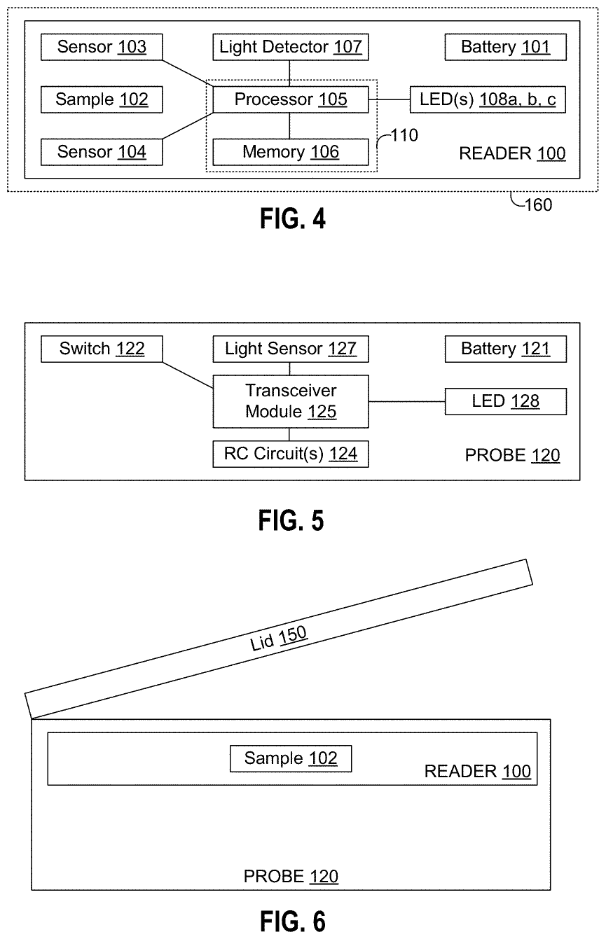

FIG. 2A shows a reader 100, according to some example embodiments. FIG. 2B illustrates an exploded view of several components of reader 100 shown in FIG. 2A. As illustrated in FIG. 2A and FIG. 2B, reader 100 comprises a housing 115. Housing 115 comprises an aperture for receiving a sample on test strip 102, one or more apertures for one or more light emitting sources, e.g., LEDs, 108a, 108b, 108c, and an aperture 107 for an ambient light sensing capability described further below. Within the housing 115, reader 100 can further comprise a battery 101, the plurality of LEDs 108a-c, an ambient light sensor proximate to the aperture 107, sample strip 102, at least one sensor 103/104, and an integrated circuit 110 comprising processing circuitry which may comprise a processor 105 and a memory 106 (shown in FIG. 4).

Battery 101 is configured to provide electrical power for any components of reader 100. In some embodiments, LEDs 108a-c can comprise an LED configured to emit red light, an LED configured to emit yellow light and an LED configured to emit green light. However, the present disclosure is not so limited and LEDs 108a-108c can be configured to emit any color or colors of light, within or outside the human-visible spectrum.

Test strip 102 is configured to receive a sample, for example an analyte suspended in an aqueous or non-aqueous solvent. In some embodiments, test strip 102 comprises a lateral flow immunoassay strip that may comprise antibodies labeled with a reflective substance, for example gold particles, latex beads, or any other suitable optically reflective or absorbing substance that is configured to migrate along test strip 102, to provide a detectable indication of the presence, absence, and/or amount of an analyte in the sample.

Sensor(s) 103/104 may be, for example, a photodetector or any other suitable detector configured to sense a characteristic (e.g., reflectivity) of a first portion of test strip 102 that changes when at least one of the liquid, analyte, and/or label migrate to the portion of test strip 102 being sensed by sensor(s) 103/104.

Light detector 107 is an optional feature configured to allow ambient light around reader 100 to reach another photodetector inside the housing 115 and communicate a signal indicative of the level of ambient light to processing circuitry 110 for waking processor 105 and/or other components of reader 100 from a low-power sleep mode. Accordingly, reader 100 can be stored in a bag 160 that is substantially opaque to the type(s) or bandwidth(s) of light that light detector 107 is configured to sense, such that, when reader 100 is disposed within bag 160 and bag 160 is sealed, reader 100 is either powered off or in a low-power sleep mode to save power and extend the useful storage life of reader 100.

Upon exiting the low-power sleep mode and transitioning to an operational mode, processing circuitry 110 may be configured to cause a first LED 108a to illuminate with a first color (e.g., green) to indicate to a user that reader 100 is ready to accept a liquid sample onto test strip 102.

The reader 100 may be configured to sample a signal from the one or more sensor(s) 103/104 according to a sampling interval (e.g., once every second) for the duration of the data collection for a test strip strip 102. In some embodiments, the sensor signals are indicative of the relative or absolute values of the amounts of light reflecting onto and/or otherwise striking the one or more sensors from sample strip 102.

Upon detection of a sample on test strip 102, either immediately after or a predetermined period of time after (e.g., 10 seconds after), the processing circuitry 110 may be configured to cause a second LED 108b to illuminate with a second color (e.g., yellow) to indicate to the user that reader 100 is analyzing test strip 102.

In some embodiments, processing circuitry 110 may cause second LED 108b to illuminate for a predetermined interval of time (e.g., 10 minutes) corresponding to a sampling interval.

At the end of the sampling interval, processing circuitry 110 may be configured to cause second LED 108b to turn off and cause third LED 108c to illuminate a third color (e.g., red). It will be appreciated that processing circuitry 110 may be configured to cause any of LEDs 108a-c to illuminate or stop illuminating in accordance with any desired information or test sequence to be delivered to a user. In some embodiments, rather than utilizing a plurality of LEDs 108a-108c, one or more LEDs or other light sources capable of multi-color illumination may be utilized instead, configured to provide the same signaling as any one or more of LEDs 108a-c.

In the interest of decreasing manufacturing costs for reader 100, the memory 106 may be significantly limited (e.g., 40 bytes). However, the present disclosure is not so limited and memory 106 can have any suitable capacity.

In some embodiments, while processor 105 may be configured to read signals from the one or more sensor(s) according to a predetermined sampling interval (e.g., once every second) for the duration of the data collection, processor 105 may be further configured to only store the Nth signal or sample from the one or more sensor(s) 103/104, where N is equal to an integer greater than one, for example and not limitation 7 or 14, and ultimately discard the remaining additional samples. Accordingly, in some embodiments, to decrease the amount of memory required to store signals and/or readings from the one or more sensors 103/104, processor 105 may be configured to store only every 7th or 14th sensor sample from each of first and second sensors 103, 104 in memory 106 and discard (e.g., not store) the rest. However, the samples that are later discarded can still be used by processor 105 in real-time or near real-time for error, variance detection and/or other signal processing functions. In some embodiments, reader 100 does not perform any computation on the signal values stored in memory 106, only storing them for future communication to probe 120 and ultimate processing by another device in the system of FIG. 1 (e.g., probe 120, computing device 140 and/or computing device 150).

As described previously, upon completion of data collection for sample strip 102, processing circuitry 110 may cause third LED 108c to illuminate. For transmitting the stored assay data to another device, processing circuitry 110 may be configured to encode sampling data stored in memory 106 as an intensity modulation of the illumination output of third LED 108c. In some embodiments, processing circuitry 110 achieves this by controlling the illumination output intensity of the third LED 108c according to a carrier frequency (e.g., 38 kHz) modulated by a serial data signal of the stored bits. Data output rate may, for example, be 1200 baud, or approximately 833 microseconds (μs) per bit. In some embodiments therefore, a 38 kHz intensity modulation of the illumination output of the LED for 833 microseconds may indicate a “1” bit and an 833 microsecond period of no intensity modulation of the LED illumination may indicate a “0” bit. A wide variety of well-known coding schemes can be used to randomize the bit stream and/or provide error detection or correction for the transmitted data. In some embodiments, processing circuitry 110 is configured to repeat the modulated readout of the data stored in memory 106 a predetermined number of times, for a predetermined amount of time, or in an infinite loop. In some embodiments, processing circuitry 110 is configured to modulate one or more escape characters (e.g., hex01 or hex10) onto the carrier frequency to signify the beginning and/or end of the data stream of bits being read out, for example, as stored in memory 106. As will be described in more detail below, probe 120 may be configured to decode the data stream modulated onto the illumination intensity of third LED 108c and forward or relay the extracted data, directly or indirectly, to computing device 140 utilizing a different mode of electronic communication such as wireless RF transmission methods including, for example, Bluetooth and WiFi.

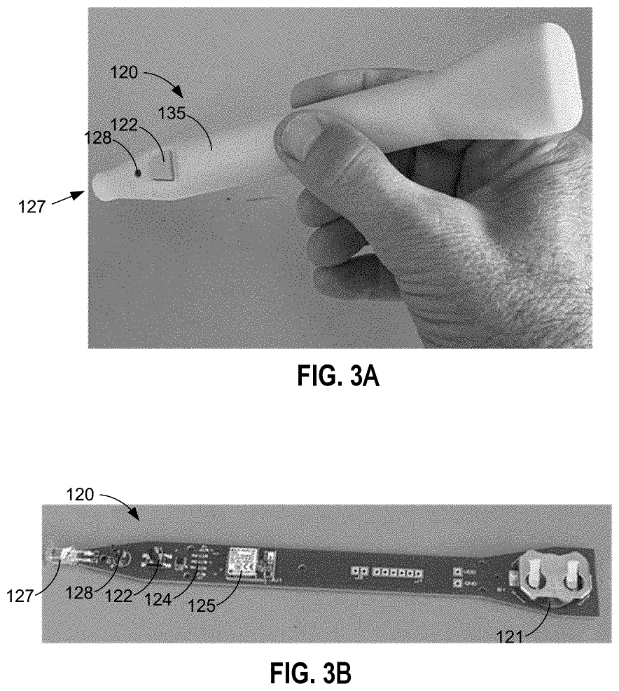

FIG. 3A shows a probe 120 for receiving data from reader 100, according to some example embodiments. FIG. 3B illustrates the probe of FIG. 3A being held by a user. FIG. 3C illustrates several components of probe 120 as shown in FIGS. 3A and 3B.

As illustrated in at least FIG. 3A, probe 120 comprises a housing 135. Housing 135 comprises an aperture for each of a light emitting source, e.g., LED 128, an aperture for a power switch or button 122 and an aperture for light sensor 127, e.g., a photodetector. As illustrated in at least FIGS. 3C and 5, within housing 135, probe 120 can further comprise a battery 121, light emitting source, e.g., LED, 128, switch 122, light sensor 127, a transceiver module 125 (e.g., a Bluetooth transceiver chip) and one or more RC circuits or chips 124 as required for operation of probe 120 as described anywhere herein.

Battery 121 is configured to provide electrical power for any components of probe 120. In some embodiments, battery 121 is replaceable. In some embodiments, LED 128 can be configured to emit any color or colors of light. Activating switch 122 turns on probe 120 and causes LED 128 to illuminate, providing visual indication that probe 120 is activated. In some embodiments, activating switch 122 also causes transceiver module 125 to transition from a low power or deep sleep mode to a functional mode. In some embodiments, in such a deep sleep mode, transceiver module 125 may be configured to draw an insignificant amount of power, e.g., 50 nanoamperes (nA), which may be less current than the intrinsic self-discharge rate of battery 121 itself. This allows for a longer battery life when probe 120 is in storage mode for an extended period of time.

Once turned on, probe 120 is configured to, for example, optically read the data being communicated by the modulated illumination intensity of LED 108c of reader 100. For example, once LED 108c on reader 100 is illuminated, a user can hold probe 120 in his or her hand and point light sensor 127 at LED 108c of reader 100. Light sensor 127 is configured to communicate a signal indicative of the illumination intensity modulation of LED 108c in reader 100 to transceiver module 125.

In some embodiments, transceiver module 125 is a Bluetooth module, for example, a programmable transceiver chip made by Nordic Semiconductor. However, any wireless communication transceiver chip capable of performing the functions described herein is also contemplated. Accordingly, transceiver module 125 may also comprise a processor, logic, memory, an antenna, an oscillator, capacitors, inductors, filters, and any other customary transceiver components as required for performing the functions described herein.

Transceiver module 125 is configured to decode the signal from light sensor 127, package and/or encode at least the decoded data (e.g., the bits as previously stored in memory 106 of microchip 110 of reader 100) into a second wireless communication format (e.g., Bluetooth) and transmit the encoded data directly, or indirectly (via WAN 130 for example), to computing device 140, which is configured to run app 145, as previously described in connection with at least FIG. 1.

Accordingly, probe 120 can act as a relay, translator and/or gateway between reader 100 and computing device 140. In some such embodiments, the data collected by reader 100 is not analyzed or substantively processed by either reader 100 or probe 120 but is, instead, collected and stored by reader 100, extracted, translated or packaged into a second communication format and relayed by probe 120, and analyzed by app 145 of computing device 140. In this way, a positive or negative result, or concentration determination of sample strip 102 is analyzed and determined by app 145 of computing device 140, rather than by reader 100 or probe 120. System design in this manner allows either or both of reader 100 and probe 120 to be made with reduced functionality and, so, with fewer components and less complication and expense. Of course, as previously described in connection with FIG. 1, at least some of this analysis could also or alternatively be carried out by an intermediate computing device 150, or similar, such as by utilizing cloud computing resources.

FIG. 6 is a block diagram for an example embodiment wherein probe 120 is configured to receive reader 100 in a predetermined orientation, according to some example embodiments. In the prior example embodiment, a user points probe 120 toward reader 100 such that light sensor 127 of probe 120 is able to sense or read data from the illumination of LED 108c of reader 100. In some other embodiments, for example as illustrated by FIG. 6, probe 120, for example housing 135 of probe 120, may be physically configured (e.g., formed) to receive reader 100, having sample strip 102 disposed therein, in such an orientation that light sensor 127 of probe 120 is facing LED 108c of reader 100. In some such embodiments, probe 120 may also have a lid 150 under which reader 100 is configured to be disposed by a user.

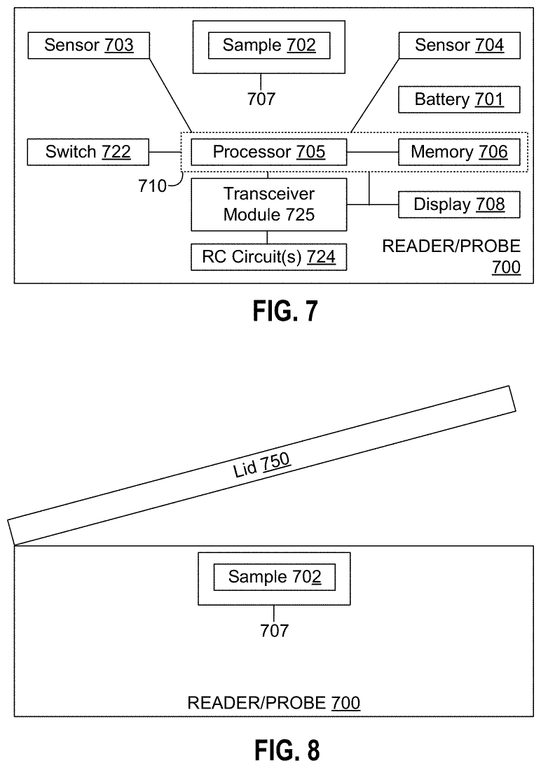

FIG. 7 is a block diagram of a single apparatus and/or housing 700 integrating several components of reader 100 and several components of probe 120, according to some example embodiments. FIG. 8 is a block diagram of a side view of apparatus 700, according to some example embodiments. As illustrated, apparatus 700 can comprise battery 701, first and second sensors 703, 704, microchip 710 including processor 705 and memory 706, switch 722, transceiver module 725, RC circuit(s) 724, and display 708, which may be substantially similar to or the same as battery 101/121, first and second sensors 103, 104, microchip 110 including processor 105 and memory 106, switch 122, transceiver module 125, RC circuit(s) 124, and LED(s) 108a-c/128, respectively, as previously described in connection with reader 100 and/or probe 120. As will be described in more detail below, in some embodiments, display 708 comprises one or more light emitting sources, e.g., LEDs, configured to independently indicate one or more states of apparatus 700 and/or configured to collectively indicate one or more states of apparatus 700.

Apparatus 700 further comprises a sample 702, which may be substantially identical to sample on test strip 102, except that, rather than being disposed within a separate reader 100, sample on test strip 702 is disposed within a plastic cassette 707 or housing that can be physically disposed within apparatus 700 during data collection and/or analysis and then discarded. Cassette 707 may be devoid of any electronics. In some such embodiments, apparatus 700 may further comprise a clampable lid 750 similar to lid 150 of probe 120 described in connection with FIG. 6, configured to snap over cassette 707. This may be different from sliding a sample strip 102, 702 into tester at least because apparatus 700 is configured to completely enclose and snap over and/or around cassette 707.

Function of apparatus 700 may be substantially as previously described for reader 100 and for probe 120 except that no aligning and optical or IR readout between a reader and a probe is necessary, since they are physically disposed within the same apparatus 700 and can communicate directly and electronically with one another. Instead, once cassette 707, having sample 702 therein, is properly disposed within apparatus 700, LED(s) 708 may be configured to function as previously described for any of LEDs 108a-c of reader 100 or LED 128 of probe 120 and the data stored in memory 706, as received from first and second sensors 703, 704, may be directly transferred from processing circuitry 710 to transceiver module 725, with or without any of the previously described carrier/data modulation and/or XOR encoding with alternating “1”s and “0”s. Moreover, the data may be transferred from processing circuitry 710 to transceiver module 725 in either real time (e.g., as the data is saved to memory 706) or in the previously-described delayed cache fashion occurring after a predetermined full data collection interval (e.g., 10 minutes).

Embodiments as described in connection with FIGS. 7 and 8 can offer a reduced cost and lower negative environmental impact compared to some other embodiments, since all that is discarded is cassette 707, comprising test strip 702 and being devoid of other electronics. All electronics previously disposed in reader 100, that would otherwise be discarded after the single use, are now disposed in apparatus 700.

In yet other embodiments, all functionality of probe 120 and/or of apparatus 700 can be disposed within and handled directly by computing device 140 on which app 145 is executed. In some such embodiments, computing device 140 can also be equipped with a camera that is configured to read blinking LED 108c. In some such embodiments, computing device 140 may be configured, similarly to the embodiments of probe 120 illustrated in FIG. 6, such that reader 100 is mountable on or in an orientation with respect to computing device 140 that allows such a camera to read blinking LED 108c. In some embodiments, reader 100, probe 120, apparatus 700 and/or computing device 140 may alternatively or additionally be configured to communicate utilizing a multilevel coding scheme that allows more complicated and/or efficient signaling patterns that incorporate simultaneous blinking and/or flashing of any two or more of LEDs 108a-c, or that blink one or more LEDs in a multicolor and/or multi-tempo signaling protocol.

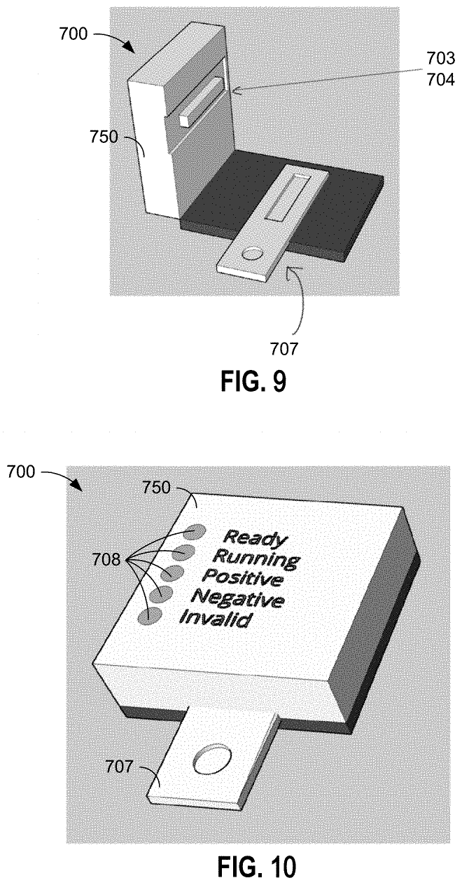

FIGS. 9 and 10 illustrate perspective views of an example embodiment of apparatus 700 and cassette 707 comprising sample 702 to be assayed. Only a subset of the components of apparatus 700 are shown for ease of illustration.

Apparatus 700 may comprise lid 750, configured to open at a hinge assembly (not shown in detail in FIGS. 9 and 10) and to accept cassette 707 in a predetermined orientation. Cassette 707 is configured to have an end portion, configured to accept a sample to be assayed, extending from apparatus 700 when cassette 707 is properly disposed at least partially within apparatus 700. In some embodiments, to facilitate proper placement of cassette 707, a base of apparatus 700 comprises a test strip mounting locations that comprises a recessed portion having a complementary shape to cassette 707. In some embodiments as illustrated in FIG. 9, the one or more sensors 703/704 may be disposed in an underside of lid 750 such that, when cassette 707 is disposed in apparatus 700 and lid 750 is closed, the one or more sensors 703/704 are disposed directly over respective portions of test strip 702 within cassette 707. In some embodiments, apparatus 700 may be configured to begin processing cassette 707 upon closing lid 750.

In FIG. 10, LEDs 708 are disposed in a top surface of apparatus 700. Each of LEDs 708 may be configured to illuminate and/or flash to indicate a different state or mode of apparatus 700. For example, one of LEDs 708 may be configured to indicate that apparatus 700 is ready to process cassette 707; one of LEDs 708 may be configured to indicate that apparatus 700 is currently processing cassette 707; one of LEDs 708 may be configured to indicate a positive result regarding the processing of cassette 707; one of LEDs 708 may be configured to indicate a negative result regarding the processing of cassette 707; one of LEDs 708 may be configured to indicate an invalid test result or an invalid cassette 707.

While FIGS. 9 and 10 illustrate a substantially square or rectangular form factor for apparatus 700, the present disclosure is not so limited and apparatus 700 may have any suitable shape and/or form factor.

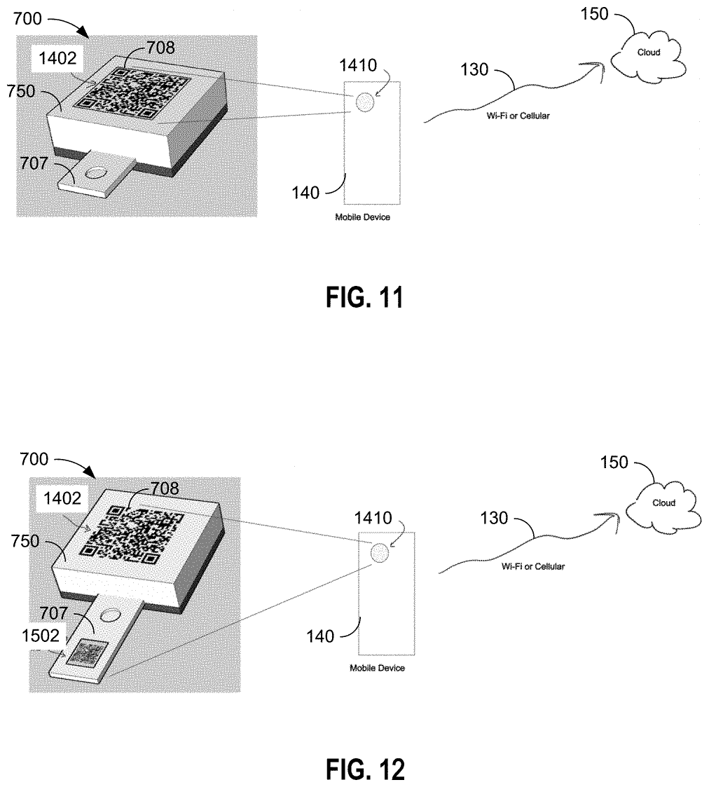

In some embodiments, apparatus 700 is configured with a display to display a 2-dimensional machine-readable code such as a QR code. For example, in embodiments according to at least FIGS. 11 and 12, display 708 of apparatus 700 may be configured to display a 2-dimensional machine-readable code 1402, for example a QR code, that can be read by a network enabled computing device such as computing device 140. The 2-dimensional machine-readable code may encode data related to the assay performance such as data related to the sampled sensor signals, e.g. raw or only partly processed sensor signals. In some embodiments, the 2-dimensional machine-readable code may encode a final assay result output indicating a result of the assay. In some embodiments, the 2-dimensional machine-readable code may additionally encode a network resource identifier such as a URL of a network service that receives the data from the computing device 140 that is encoded in the 2-dimensional machine-readable code. The accessed network service may process the received assay data to generate a final assay result output and incorporate that result in a webpage delivered back to the computing device 140 by the network service so that a result of the test may be displayed to a user. In some embodiments, the QR code may include a reader ID (e.g., an identification of apparatus 700) and/or the result and/or value of a test performed on cassette 707.

In embodiments according to FIG. 12, cassette 707 may also comprise a code, such as a second QR code 1502 configured to identify, for example, a type of test cassette 707 is configured for (e.g., a test identifier regarding any one or more of bedbugs, ticks, lice, etc.), a duration of the test of cassette 707, a positive and/or negative threshold for the test of cassette 707, a serial number, a lot number, an expiration date, and/or a hash code to ensure cassette 707 is genuine and not counterfeit.

A system according to either of FIGS. 11 and 12 may be substantially similar to the system of FIG. 1, however, including integrated apparatus 700 as described anywhere herein rather than separate reader 100 and probe 120. Mobile device 140, which may be a mobile phone or tablet for example, comprises a camera 1410 configured to capture an image of the code 1402 on apparatus 700 (see FIGS. 11 and 12) and/or a code 1502 on cassette 707 (see FIG. 12).

Where camera 1410 is configured to capture an image of code 1502 on cassette 707, mobile device 140 may be configured to extract any of the previously-mentioned information encoded in code 1502. In addition, and/or alternative, mobile device 140 may be configured to extract a subset of the previously mentioned information encoded in code 1502 and derive a different subset of the previously mentioned information, or other information, that may not be encoded in code 1502. For example, code 1502 may encode a serial number of cassette 707 and mobile device 140 may be configured to extract the serial number from code 1502 and then derive, request from another computing device 150 over a network 130 or look up a lot number and/or expiration date based on the serial number. As another example, code 1502 may encode a test identifier of cassette 707 and mobile device 140 may be configured to extract the test identifier number from code 1502 and then derive, request from another computing device 150 over a network 130 or look up a duration of the test and/or positive and/or negative thresholds for the test based on the test identifier. In some embodiments, apparatus 700 may be configured to read or extract information directly from cassette 707 once disposed therein, for example, via one or more electrical contacts communicatively coupling circuitry in cassette 707 and circuitry in apparatus 700 and/or via another sensor configured to read a pattern or code on cassette 707. Once mobile device 140 and/or apparatus 700 has extracted, derived, received via request or looked up sufficient information, apparatus 700 may perform a test on cassette 707.

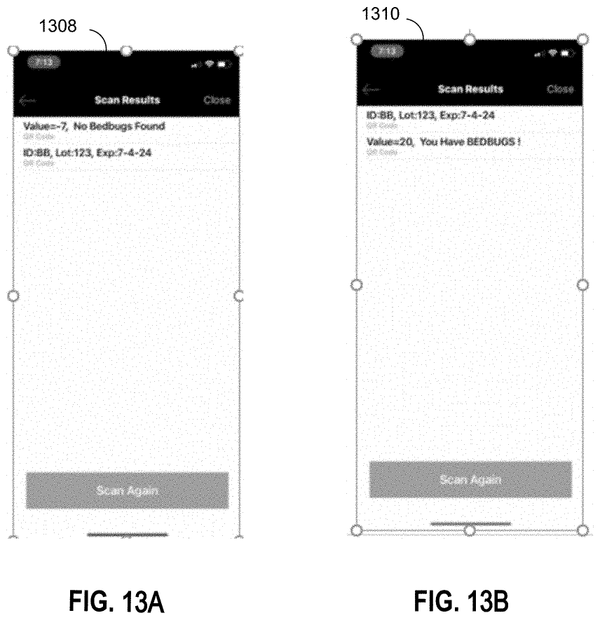

FIG. 13A illustrates a screen shot of app 145 returning another set of information extracted, derived, received via request or looked up based on the two codes (e.g., 1402, 1502). The cassette ID, lot number, and expiration date are illustrated in the first entry, while a different result of the test on cassette 707 is illustrated in the second entry.

FIG. 13B illustrates another screen shot of app 145 returning yet another set of information extracted, derived, received via request or looked up based on the two codes (e.g., 1402, 1502). A result of the test on cassette 707 is illustrated in the first entry, while the cassette ID, lot number, and expiration date are illustrated in the second entry.

Example embodiments of apparatus 700 and/or of cassette 707 are illustrated in more detail in one or more of the following FIGs.

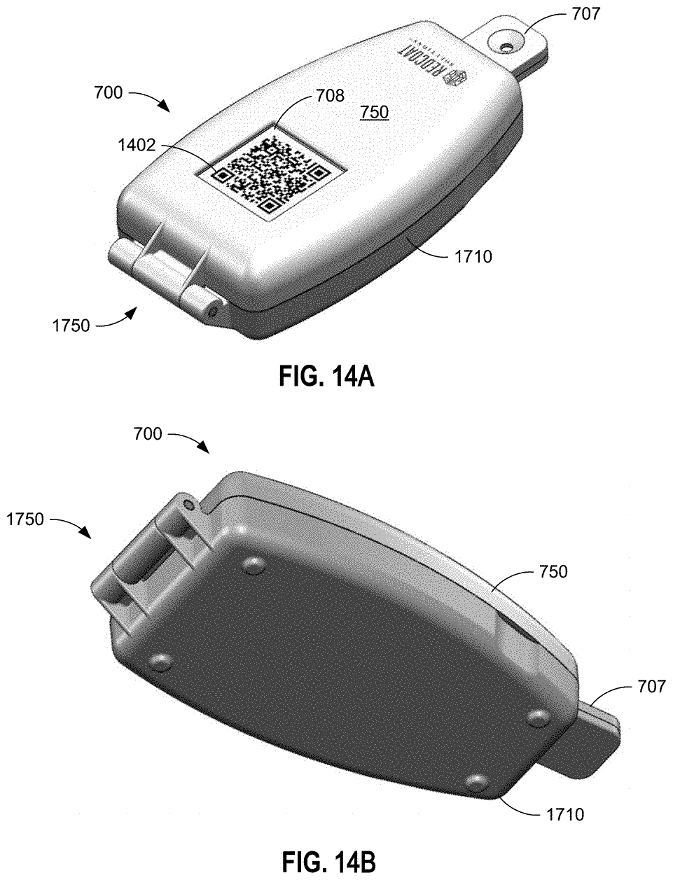

FIG. 14a and FIG. 14B illustrate an apparatus 700 having cassette 707 disposed therein. Apparatus 700 comprises a housing base 1710 and lid 750 coupled to one another via a hinge assembly 1750. Display 708 is illustrated displaying QR code 1402, visible through an aperture in lid 750. An advantageous display technology for this application is known as e-paper, which is a very low power consuming persistent display. However, it will be appreciated that any display technology could be utilized. FIG. 14B illustrates a perspective bottom view of apparatus 700.

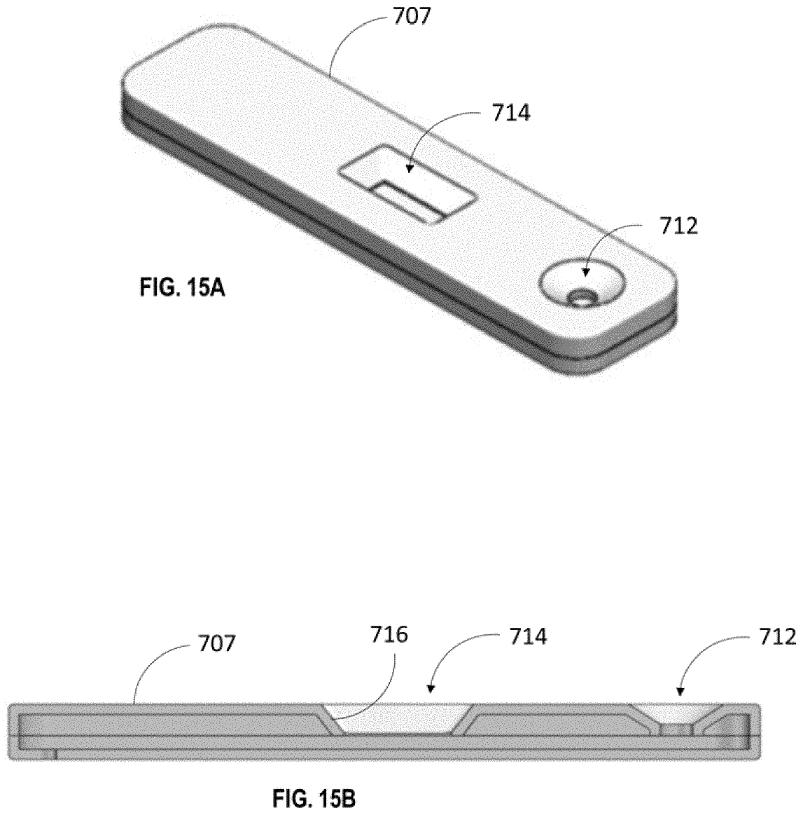

FIGS. 15A and 15B illustrate an example embodiment of a test strip cassette 707. In this embodiment a housing includes a sample application opening 712 and a test result viewing opening 714. As can be seen in FIG. 15, the test result viewing opening comprises beveled walls at least partially around the edge of the opening, a function of which is described further below. The housing may be formed from a top portion and an opposing bottom portion.

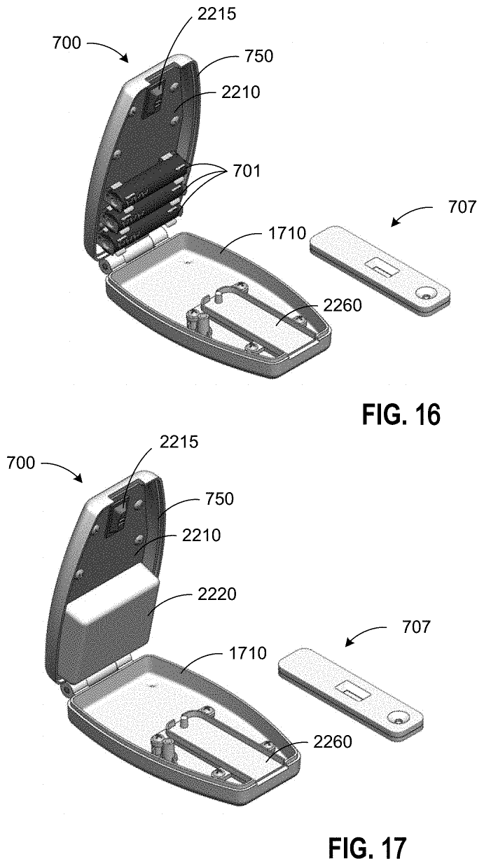

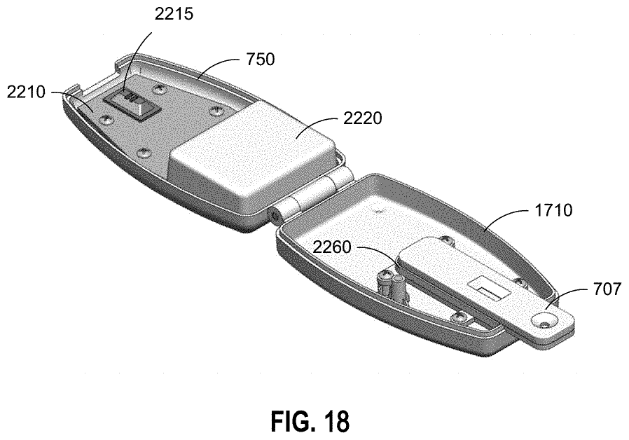

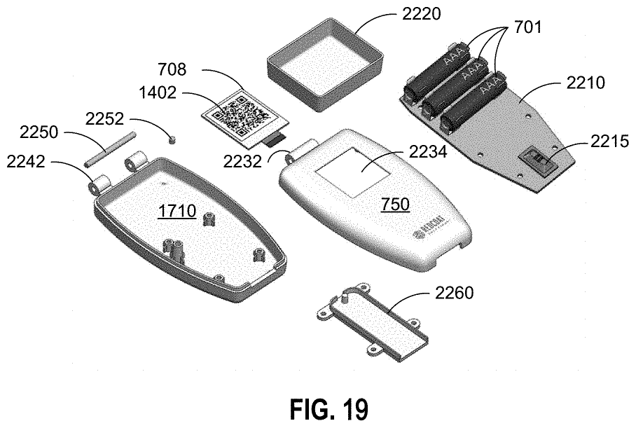

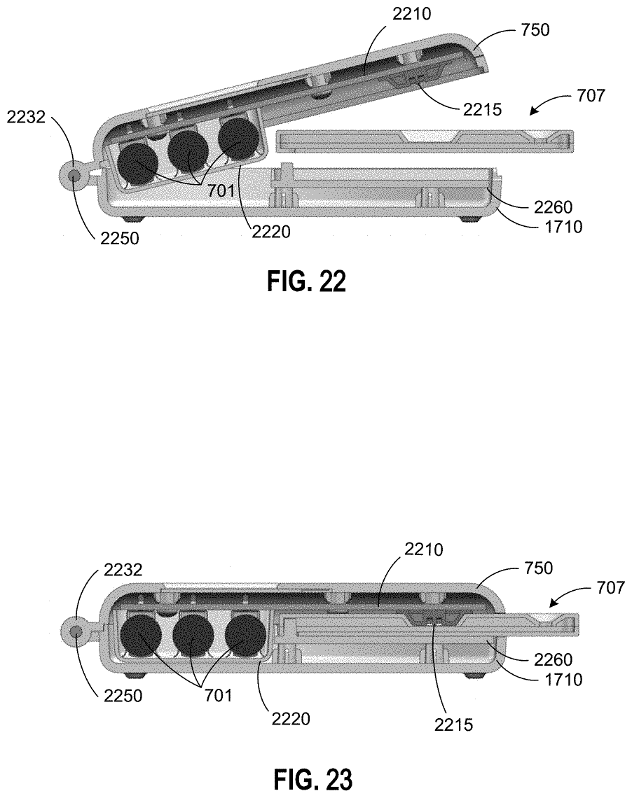

Referring now to FIGS. 16 through 23, lid 750 comprises aperture 2234 (FIG. 19) through which display 708 and QR code 1402 is ultimately made visible. In some embodiments, a printed circuit board (PCB) 2210 is configured to be disposed on an underside of lid 750. While PCB 2210 may comprise any electronics and/or circuitry of apparatus 700 as described anywhere herein, FIG. 16 specifically illustrates one or more sensors 703/704 and batteries 701 (e.g., 3× AAA batteries) disposed thereon. Battery cover 2220 is configured to be disposed over a top (or bottom) and sides of batteries 701. For example, FIG. 16 illustrates apparatus 700 with PCB 2210 attached to the underside of open lid 750, cassette mounting structure 2260 attached to base shell 1710, and neither cassette 707 nor cover 2220 coupled to apparatus 700. FIG. 17 illustrates apparatus 700 as in FIG. 16 but with cover 2220 coupled over batteries 701. FIG. 18 illustrates apparatus 700 as in FIG. 17 but lid laid open, completely flat and cassette 707 properly disposed on cassette mounting structure 2260. As illustrated, one or both of PCB 2210 and cassette mounting structure 2260 may be respectively secured to lid 750 and base shell 1710 using screws.

FIG. 19 illustrates an exploded perspective view of apparatus 700 of any of FIGS. 14-18, according to some example embodiments. Apparatus 700 comprises base housing portion 1710 having a first portion 2242 of hinge apparatus 1750 and lid housing portion 750 having a second portion 2232 of hinge apparatus 1750. A hinge pin 2250 may be configured to pass through apertures in each of first and second portions 2242, 2232 of hinge apparatus 1750. In some embodiments, as illustrated in FIG. 19, second portion 2232 may be configured to be disposed between each of two laterally disposed aspects of first portion 2242.

Base shell 1710 comprises a cassette mounting structure 2260 configured to receive cassette 707 such that at least an end portion of cassette 707 extends outside of apparatus 700.

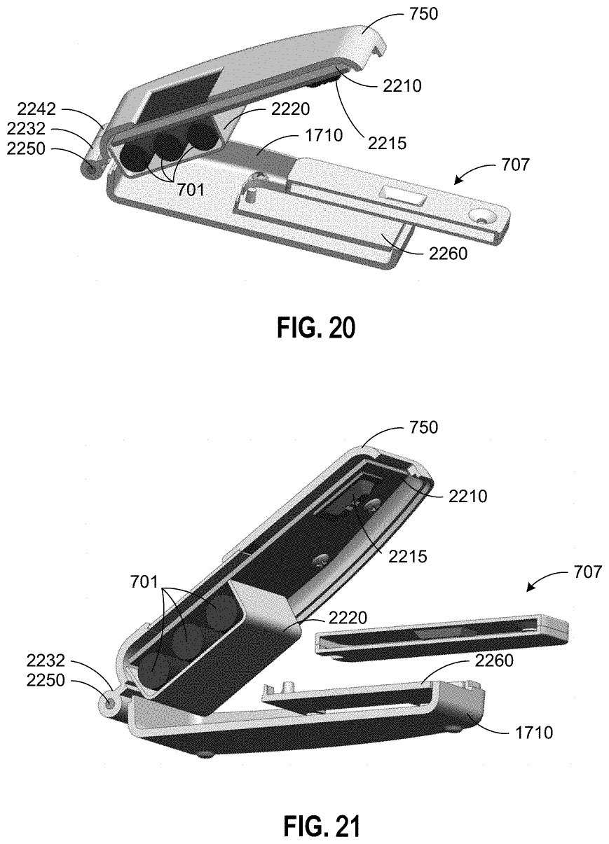

FIGS. 20-23 illustrate different cutaway views of apparatus 700 with all aspects of apparatus 700 installed. A protruding housing 2215 forms a mounting location for the one or more sensors 703/704 which is disposed on PCB 2210 (which is coupled to the underside of lid 750).Cassette mounting structure 2260 is disposed on base housing portion 1710 such that when cassette 707 is disposed in structure 2260 and lid 750 is closed, the one or more sensors 703/704 are aligned directly over the correct portion of test strip 702 to be analyzed within cassette 707.

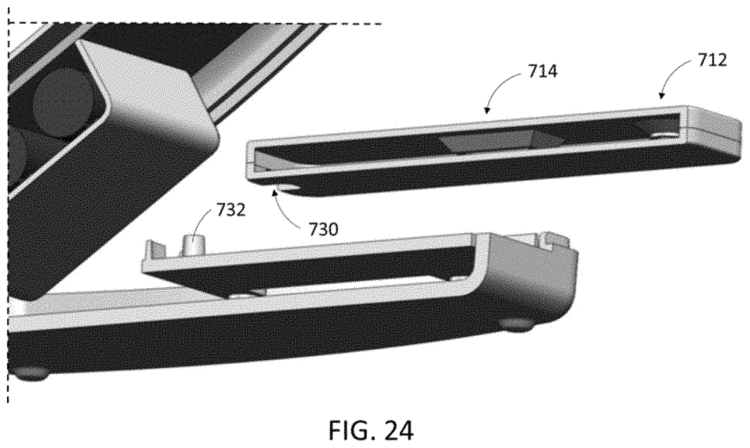

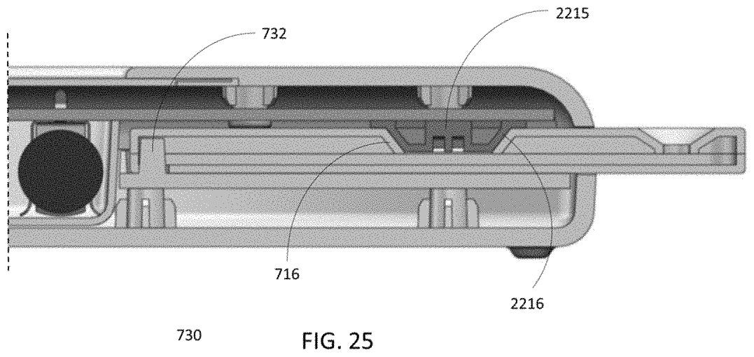

FIG. 24 and FIG. 25 illustrate the placement and registration of the cassette 707 with respect to the test strip/cassette mounting structure 2260 As shown in FIG. 24, the mounting structure includes a protruding pin 732 that mates with an opening 730 in the cassette 707 configured to accept the pin 732 when the cassette 707 is placed and oriented correctly on the mounting structure 2260. FIG. 25 shows the pin 732 mated with the opening 730. The pin 732 and opening 730 form registration or alignment features for the cassette 707 with respect to the mounting structure 2260.

In addition, as shown in FIG. 25, the sensor housing 2215 and the test result viewing opening 714 are also configured to mate in a manner providing accurate registration/alignment of the one or more sensors with the desired location of the test strip. In this embodiment, the viewing opening 714 on the cassette 707 has one or more inwardly beveled walls 716 around at least a portion of the viewing opening 714. The mounting structure 2215 for the one or more sensors has one or more protruding walls 2216 with matching bevels to mate snugly inside the result viewing opening 714. These registration and alignment structures, which can be provided together or separately in various embodiments, help ensure that a user of the device obtains accurate and reproducible test strip and sensor alignments for each assay performed, enhancing accuracy of test results.

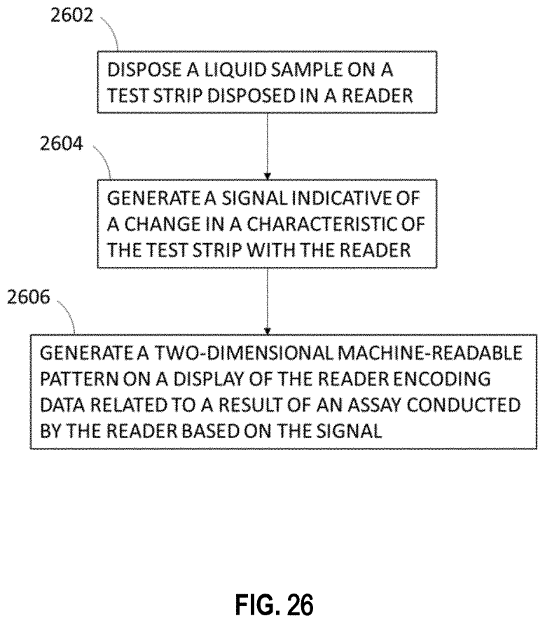

FIG. 26 illustrates a flowchart of another method for testing an assay and reading out a result of such testing, according to some example embodiments. The method of FIG. 26 may correspond with or apply to any apparatus described in this disclosure.

Block 2602 includes disposing a liquid sample on a test disposed in a reader. Test strip 702 may be disposed in a cassette 707 that is removably disposed in apparatus 700.

Block 2604 includes generating a signal indicative of a change in a characteristic of the test strip with the reader.

Block 2606 includes generating a pattern on a display of the reader, the pattern encoding data related to a result of an assay conducted by the reader based on sampled sensor signals. For example, as previously described, display 708 may generate a pattern (e.g., a bar code or QR code. The pattern encodes data related to a result of an assay conducted by apparatus 700.

In some embodiments, the method further include utilizing an application running on a mobile device to cause a camera to generate an image of the pattern on the display of the reader and decode the data related to the result based on the image. For example, as previously described, app 145 running on mobile device 140 may cause camera 1410 to generate an image of pattern 1402 on display 708 of apparatus 700 and decode the data related to the result based on the image.

In some embodiments, app 145 may cause camera 1410 to generate the image that simultaneously includes second pattern 1502 on cassette 707 and pattern 1402 on display 708 of apparatus 700. App 145 may further decode the second data based on the image.

In some embodiments, apparatus 700 may read information directly from cassette 707 via one or more electrical contacts communicatively coupling cassette 707 with apparatus 700 when cassette 707 is disposed on cassette carrier 2260 within apparatus 700.

In some embodiments, sample strip 702 comprises a reflective substance (e.g., gold) and the characteristic being sensed is a reflectivity of the sample strip. In some embodiments, the first portion of sample strip 702 is disposed between the initial portion and the second portion of the sample strip. The liquid sample received by sample strip 702 is configured to migrate from the initial portion toward the first and second portions of sample strip 702. In some embodiments, apparatus 700 includes hinged lid 750 and first and second sensors 703, 704 are disposed on an underside of lid 750 such that, when cassette 707 is disposed in carrier 2260 and lid 750 is closed, first and second sensors 703, 704 are disposed directly over the respective first and second portions of sample strip 702 within cassette 707. In some embodiments, first and second sensors 703, 704 are disposed on PCB 2210 secured to an underside of lid 750 such that first and second sensors 703, 704 protrude from PCB 2210 and into one or more recesses 2324 in cassette 707 when cassette 707 is disposed on carrier 2260. In some embodiments, the data related to the result of the assay includes at least one of text of a result of the assay, a pictorial representation of the result of the assay, and a link to a webpage configured to convey at least one of a result of the assay, and a unique identifier of apparatus 700. In some embodiments, cassette 707 comprises second pattern 1502 encoding second data related to at least one of a type of assay cassette 707 is configured for, a duration for carrying out the assay, a positive and/or negative threshold associated with the assay, a unique serial number, lot number and/or expiration date of cassette 707, and a hash code.

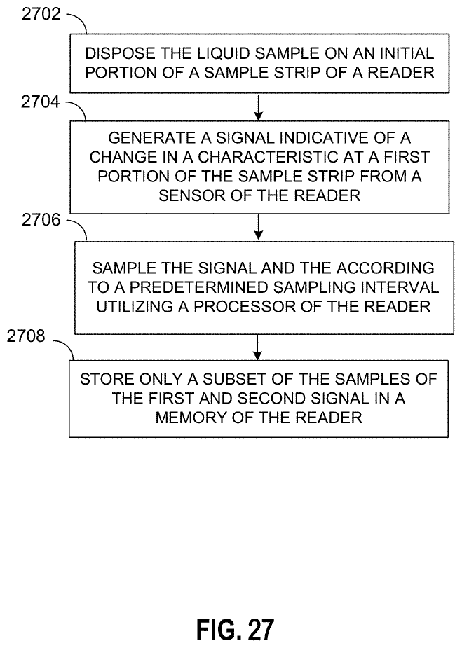

FIG. 27 illustrates a flowchart of a method for testing an assay and reading out a result of such testing, according to some example embodiments. The method may correspond with or apply to any apparatus described in this disclosure.

Block 2702 includes disposing the liquid sample on a portion of test strip of a reader. For example, as previously described, a user can dispose the liquid sample on an initial portion of sample strip 102 of reader 100.

Block 2704 includes generating a signal indicative of a change in a characteristic at a portion of the test strip from a sensor of the reader. In some embodiments, the characteristic is a reflectivity of sample strip 102.

Block 2706 includes sampling the signal according to a predetermined sampling interval utilizing a processor of the reader. For example, processor 105 of processing circuitry 110 of reader 100 is configured to sample a signal from the one or more sensors according to a predetermined sampling interval (e.g., once every second) for the duration of the data collection for sample strip 102.

Block 2708 includes storing only a subset of the samples of the signal in a memory of the reader. For example, memory 106 of processing circuitry 110 of reader 100 can be configured to only store the Nth signal or sample from each of first and second sensors 103, 104, where N is equal to an integer greater than one, for example and not limitation 7 or 14.

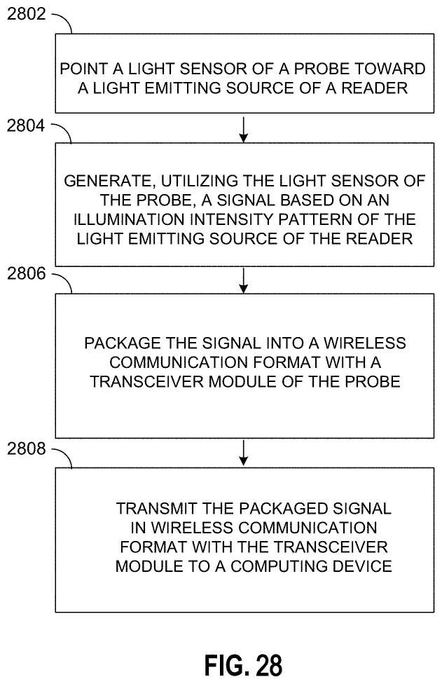

FIG. 28 illustrates a flowchart of another portion of a method for performing an assay and reading out a result of such testing, according to some example embodiments. The method of FIG. 28 may correspond with or apply to any apparatus described in this disclosure. In some embodiments, the method of FIG. 28 can follow the method of FIG. 27.

Block 2802 includes pointing a light sensor of a probe toward a light emitting source of a reader. For example, a user can point light sensor 127 of probe 120 toward LED 108c of reader 100. In some embodiments, for example as previously described in connection with FIG. 6, block 2802 can also include physically disposing reader 100 into probe 120 in a predetermined orientation such that light sensor 127 of probe 120 is facing LED 108c of reader 100.

Block 2804 includes generating, utilizing the light sensor of the probe, a signal indicative of the subset of the samples based on an illumination intensity pattern of at least the first light emitting source of the reader. For example, light sensor 127 of probe 120 can be configured to generate a signal indicative of the subset of samples based on the blinking pattern of LED 108c of reader 100.

Block 2806 includes packaging the signal indicative of the subset of the samples into a predetermined wireless communication protocol format utilizing a transceiver module of the probe. For example, transceiver module 125 of probe 120 can be configured to package the signal indicative of the subset of the samples generated by light sensor 127 of probe 120 into a predetermined wireless communication protocol format. In some embodiments, the predetermined wireless communication protocol is Bluetooth.

Block 2808 includes transmitting the packaged signal in the predetermined wireless communication protocol format, utilizing the transceiver module, to a computing device configured to analyze the subset of the samples. For example, transceiver module 125 of probe 120 can be configured to transmit the packaged signal in the predetermined wireless communication protocol format to computing device 140 (and/or intermediate computing device 150) configured to analyze the subset of the samples ultimately relayed from memory 106 of reader 100, through probe 120, to computing device 140 and/or intermediate computing device 150.

In some embodiments, neither reader 100 nor probe 120 is configured to analyze or substantively process the subset of the samples of the first and second signals initially generated by first and second sensors 103, 104 of reader 100 with respect to determination of a result of the assay of the liquid sample on sample strip 102.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

1. A liquid sample assay device comprising:
a first housing portion;
a second housing portion;
a test strip mounting location associated with at least one of the first housing portion and the second housing portion;
a user operable coupling configured to selectively allow open and closed configurations of the first housing portion with respect to the second housing portion;
wherein the coupling is configured to allow a user to place the first housing portion and the second housing portion in the open configuration to the expose the test strip mounting location, place a test strip at the test strip mounting location, and place the first housing portion and the second housing portion in the closed configuration for performing an assay of the test strip.

2. The liquid sample assay device of claim 1, wherein the first housing portion forms a lid configured to cover the second housing portion with the test strip mounting location positioned between the first housing portion and the second housing portion.

3. The liquid sample assay device of claim 1, wherein the coupling comprises a hinge.

4. The liquid sample assay device of claim 1, wherein the test strip mounting location is configured to engage with a housing of a test strip cassette.

5. The liquid sample assay device of claim 4, wherein the test strip cassette is a disposable single use test strip cassette.

6. The liquid sample assay device of claim 1, comprising one or more sample sensors associated with at least one of the first housing portion and the second housing portion.

7. The liquid sample assay device of claim 1, wherein the liquid sample assay device forms a hand-held housing.

8. The liquid sample assay device of claim 1, wherein the test strip mounting location is configured to receive a test strip without sliding.

9. The liquid sample assay device of claim 1, comprising a display associated with at least one of the first housing portion and the second housing portion.

10. (canceled)

11. A device for assaying a liquid sample, the device comprising:
a housing configured to receive a test strip in a predetermined orientation such that a portion of the test strip is disposed within the apparatus and a portion of the test strip extends outside of the apparatus;
at least one sensor in the housing configured to generate a signal indicative of a change in a characteristic of the sample strip; and
a display configured to generate a machine-readable pattern encoding data related to the signal.

12. The device of claim 11, wherein the housing comprises a hinged lid.

13. The device of claim 12, wherein the at least one sensor is mounted to an underside of the hinged lid.

14. The device of claim 13, wherein the at least one sensor is disposed on a printed circuit board secured to an underside of the lid.

15. The device of claim 14, wherein the at least one sensor protrudes from the printed circuit board.

16. The device of claim 11, wherein the display comprises an e-paper display.

17. The device of claim 11, wherein the machine-readable pattern comprises a non-human readable pattern.

18. The device of claim 11, wherein the machine-readable pattern comprises a QR code.

19. The device of claim 11, wherein the data related to the signal comprises a final assay output.

20-23. (canceled)

24. A method for assaying a liquid sample, the method comprising:
disposing the liquid sample on a portion of a test strip;
placing a portion of the test strip into a reader comprising a sensor and a display;
generating a signal indicative of a change in a characteristic of the test strip with the sensor; and
generating a machine-readable pattern on the display encoding data related to the signal.

25. The method of claim 24, comprising reading the machine-readable pattern with a handheld device.

26. The method of claim 25, comprising displaying a final assay output to the user on a display of the handheld device.

27-31. (canceled)
